(19) United States
(12) Patent Application Publication
(10) Pub. No.: US 2023/0174593 A1
(43) Pub. Date: Jun. 8, 2023

(54) ARTIFICIALLY LINKED TANDEM ACYL CARRIER PROTEINS TO ENHANCE FATTY ACID PRODUCTION

(71) Applicant: UNIVERSITY OF PUERTO RICO, San Juan, PR (US)

(72) Inventors: Carlos Ralph RULLAN-LIND, San Juan, PR (US); Abel Juan BAERGA-ORTIZ, San Juan, PR (US)

(21) Appl. No.: 17/636,470

(22) PCT Filed: Aug. 18, 2020
(86) PCT No.: PCT/US2020/046770

§ 371 Date: Feb. 18, 2022

Related U.S. Application Data
(60) Provisional application No. 62/889,892, filed on Aug. 21, 2019.

(51) Int. Cl. C07K 14/245; C07K 14/28

(52) U.S. Cl. CPC C07K 14/245; C07K 14/28; C07K 2319/00

(57) ABSTRACT

The disclosure provides artificially linked acyl earner proteins that enhance fatty acid biosynthesis and methods for mating the artificially linked acyl earner proteins. The fused dimer comprises acyl carrier proteins and peptide linkers.

### CROSS-REFERENCE TO RELATED APPLICATIONS

This application is a U.S. National Phase of International Application No. PCT/US2020/046770, filed Aug. 18, 2020, which claims priority to U.S. Provisional Application No. 62/889,892, filed Aug. 21, 2019, both of which are incorporated by reference herein in their entirety.

### STATEMENT REGARDING FEDERALLY SPONSORED RESEARCH

This invention was made with government support under award numbers CHE0953254 awarded by the National Science Foundation and R25M061838 awarded by the National Institute of Health. The government has certain rights in the invention.

### REFERENCE TO AN ELECTRONIC SEQUENCE LISTING

The instant application contains a Sequence Listing which has been submitted electronically as a text file in ASCII format that is 3 kb in size, was created on Aug. 13, 2020, and is hereby incorporated by reference in its entirety. The name of the ASCII text file is “19-1313-WO-US_Sequences_ST25_FINAL.txt.”

### FIELD OF THE INVENTION

The present disclosure relates to biosynthesis of fatty acids. More specifically, the disclosure relates to the artificial linkage of single-domain acyl carrier proteins (ACPs) in order to increase production of fatty acids. The disclosure further relates to methods for linking single-domains ACPs.

### BACKGROUND

Fatty acid (FA) biosynthesis in bacterial and microbial cultures offers a promising solution to sustainable generation of biofuels and biomaterials. For example, FA production in Escherichia coli (E. coli) has the advantages of low environmental impact, short production times, and ease of genetic manipulation. Consistent with this, free fatty acids produced in the metabolic pathways of bacteria such as E. coli can be used, for instance, as precursors for biofuels (Magnuson et al., 1993; Microbiol. Rev. 57, 522-542).

FA biosynthesis is a ubiquitous pathway across organisms of varied phylogenies with acyl carrier proteins (ACPs) being central to the production of fatty acids. (Cronan & Thomas, Methods Enzymol. 459, 395-433 (2009)). FA biosynthesis requires concerted action of multiple enzymes that catalyze condensation of malonyl-CoA units, resulting in a fatty acyl chain that becomes modified by ketoreductase, dehydratase, and enoyl reductase enzymes (Heath and Rock, 1996, J. Biol. Chem. 271, 27795-27801). During biosynthesis of FAs, all starting materials and intermediates are covalently linked to the free thiol of the 4′-phosphopanthetheine prosthetic group in the ACP domain which serves as a shuttle from one active site to the next (FIG. 1). ACPs are also important and serve homologous functions in other metabolic pathways such as the biosynthesis of polyketides and non-ribosomal peptides by enzyme systems that employ a molecular logic similar to that of fatty acid synthases (Huang et al., 2016, Org. Lett. 18, 4288-4291). While ACP functions as an independent protein in many organisms, there are species that contain covalently linked ACP domains in tandem that allow for chemical modification via an acyl unit, as building blocks of polymers, for example for fatty acids and polyketides as well as other functions (Magnuson et al., 1993, Microbiol. Rev. 57, 522-542; Metz et al., 2001, Science 293, 290-293).

The presence of tandem ACP domains is present in synthase systems involved in production of several vital products, for example, the polyunsaturated fatty acid (PUFA) synthases, and polyketide synthases (PKSs) (Hopwood & Sherman, 1990, Annu. Rev. Genet. 24, 37-66; Hopwood, 1997, Chem. Rev. 97, 2465-2498; Okuyama et al., 2007, Appl. Environ. Microbiol. 73, 665-670). Many species of PUFA-producing marine bacteria contain between 2-9 tandem (FIG. 2) ACP domains in their PUFA synthases, as seen in Photobacterium profundum synthase which contains 5 covalently linked ACP domains (FIG. 3) (Okuyama, et al., 2007, Appl. Environ. Microbiol. 73, 665-670 (2007); Metz et al., 2009, Plant Phvsiol. Biochem. 47, 472-478). Examples of PKSs with tandem ACP domains have also been identified in numerous organisms including Pseudomonas fluorescens, although most known PKSs contain a single ACP domain for each cycle of chain elongation (El-Sayed et al., 2003, NCIMB 10586. Chem. Biol. 10, 419-430; Fujii et al., 2001, Chem. Biol. 8, 189-197; Rahman et al., 2005, J. Biol. Chem. 280, 6399-6408).

The advantages of tandem ACP arrangement in synthase systems have been reported for both biosynthesis of FAs and polyketides. The effect of tandem ACP domains is proportional increases in PUFA yields to the number of functional ACPs (Jiang et al., 2008,. J. Am. Chem. Soc. 6336-6337). Further, ACP domains are functionally equivalent regardless of their physical location (Id.).

The structure and number of ACP domains control PUFA productivity as revealed by the increased PUFA yields with the insertion of either inactivated or overly active ACP domains Hayashi et al., 2016, Enhanced production of polyunsaturated fatty acids by enzyme engineering of tandem acyl carrier proteins. Sci. Rep. 6, 35441). The PfaA subunits which harbor ACP domains and their linkers are exchangeable between organisms and can influence the FA yields in a number of organisms (Id.). In addition, the production of polyketide antibiotics also benefits from the presence of tandem ACPs. The production of the clinical antibiotic mupirocin can increase antibiotic production with tandem ACPs by increasing pathway flow, or mupirocin production as a result of either a parallel or in series arrangement (Rahman et al., 2005, J. Biol. Chem. 280, 6399-6408). Previous work elucidated the solution structure of tandem ACPs from P. profundum, demonstrating that they adapt an elongated beads-on-a-string arrangement with flexible linkers and relatively independent domains (Trujillo et al., 2013, PLoS ONE 8, e57859).

In E. coli, ACP is an acidic, 78 amino acid protein, encoded by the acpP gene (Rawlings & Cronan, 1992, J. Biol. Chem. 267, 5751-5754). ACP importance is reflected in its abundance, composing 0.25% of all soluble proteins in E. coli (Rock & Cronan. 1979, J. Biol. Chem. 254, 9778-9785). ACP and ACP-bound intermediates are used in the fatty acid biosynthetic pathway, while degradation is specific towards CoA-acyl chains (JanBen & Steinbiichel, 2014, Biotechnol. Biofuels 7, 7). Thus, aside from its role as a shuttle for fatty acyl intermediates, ACP also plays a part in the regulation of the anabolic/catabolic pathways. As an active monomer, ACP is rod-shaped, and adopts a four-helix bundle fold which allows electrostatic interactions with other fatty acid enzymes (Nguyen et al., 2014, Nature 505, 427-431; Zhang et al., 2016, Cell Res. 26, 1330-1344). X-Ray crystallography and NMR studies comparing E. coli ACP to ACP from other organisms reveal that the four α-helix bundle and hydrophobic interactions that stabilize the bundle are structurally similar (Wong et al., 2002, J. Biol. Chem. 277, 15874-15880; Xu et al., 2001, Struct. Lond. Engl. 1993 9, 277-287; Holak et al., 1998, Biochemistry (Mosc.) 27, 6135-6142). No studies have reported oligomerization of E. coli ACP.

Given the importance of ACP in fatty acid production, a common strategy to enhance fatty acid yields is to overexpress ACP. However, this strategy has proven unviable as overproduction of native E. coli ACP, is toxic the host (Keating et al., 1995, J. Biol. Chem. 270, 22229-22235). Interestingly, the expression of ACP from Azospirillum brasilense in E. coli increased the production of octadecenoic acid (18:1) 2-fold, demonstrating the validity of a fatty acid enhancement strategy based on the optimization of ACP (Jha et al., 2007, Plant Physiol. Biochem. 45, 490-500).

An advantage of the current application is the use of artificially linking of ACP domains as a means to optimize fatty acid production. This is advantageous over current methods that increase production of native E. coli. Consistent with this, herein we describe ACP domains from E. coli artificially linked using the naturally occurring linker from PfaA from Photobacterium profundum (FIG. 4). By mimicking the tandem ACP arrangement seen in PUFA and polyketide synthases we are able to increase fatty acid yields of this fused tandemization.

### SUMMARY

The present disclosure provides artificially linked acyl carrier proteins (ACPs) to enhance fatty acid production in a host. The ACPs are artificially linked via peptide linkers from P. profundum. The disclosure also provides methods for making artificially linked ACPs.

In a first embodiment, the disclosure provides ACPs, comprising a population of ACP and a population of linkers, wherein the population of ACPs are artificially covalently linked via the population of peptide linkers.

In a further embodiment, the ACPs are artificially linked via the population of peptide linkers to form a linked tandem ACP. In one aspect, the linked tandem ACPs is arranged in a multi-domain arrangement. In yet another aspect the linked ACP multi-domain arrangement is a flexible multi-domain arrangement. In a further aspect the multi domain arrangement is a beads-on-a-string arrangement of domains.

In another embodiment the ACP are isolated from a microbial host. In one aspect the microbial host is Escherichia coli. In a third embodiment the population of peptide linkers is isolated from a bacteria. In one aspect the bacteria is P. profundum.

In yet another embodiment the ACPs exhibit increased fatty acid biosynthesis when compared to a single-domain ACP.

In another embodiment is a method of making artificially linked ACPs comprising the steps of: constructing artificially linked multi domain ACP fragments; cloning and expressing the artificially linked multi domain ACP fragments to create artificially linked multi domain ACP proteins; purifying a first plurality of artificially linked multi domain ACPs and quantifying fatty acid yield of a second plurality of artificially linked multi domain ACPs.

In one aspect the population of ACPs is isolated from a microbial host. In another aspect the microbial host is E coli. In still another aspect, a population of peptide linkers is isolated from bacteria. In yet another aspect the bacteria is P. profundum.

In another embodiment the artificially linked multi domain ACP fragments are constructed by artificially linking the population of ACP genes via the population of peptide linker genes in tandem. In one aspect, the artificially linked multi domain ACP fragments are cloned and expressed in a microbial cell. In yet another aspect, the microbial cell is an E. coli cell. In still another aspect the artificially linked multi domain ACP protein constructs are purified based on size and affinity.

In yet another embodiment the pair distribution function (P(r)) of the artificially linked multi-domain ACP protein constructs is a beads-on-a-string arrangement of domains

In still another embodiment, the artificially linked multi-domain ACP protein constructs are expressed in a microbial culture. In one aspect, the microbial culture is E. coli. and fatty acid yields determined at a plurality of temperatures.

### BRIEF DESCRIPTION OF THE DRAWINGS

FIG. 1. ACP binding of fatty acids during catalytic steps of fatty acid synthesis. During each cycle of fatty acid synthesis, fatty acids remain bound via a thioester bond to the 4′-phosphopanthetheine prosthetic group of ACP.

FIG. 2. Evolutionary conservation of tandem ACPs. Tandem ACPs are a common occurrence in PUFA-producing marine bacteria. Most PUFA synthase multienzymes contain between 2-9 ACP domains in tandem. In contrast, E. coli uses a single type II ACP for fatty acid production.

FIG. 3. Fatty acid type I systems as a model for producing E. coli tandem ACPs that mimics the tandem architecture observed in P. profundum ACP.

FIG. 4. Representation of linked native E. coli ACP into tandem constructs using the linker region from the P. profundum PfaA multi-domain enzyme.

FIG. 5. Schematic for the preparation of fused tandem ACPs. Primers for the amplification of acpP contained additional sequence complementary to either the 5′ or 3′ terminus of the sequence for the pfaA linker.

FIG. 6. Generation of tandem acpP gene constructs. Artificial genes were visualized on a 2% agarose gel. A band in the 552 bp region was observed corresponding to acpP₂ and acpP₃ was observed as a band in the 870 bp region. Both bands were purified by gel extraction and cloned into pET200/D-TOPO. Plasmids were sent for sequencing and verified by the Sanger method.

FIG. 7. Proteins were purified by NiNTA followed by size exclusion chromatography. The chromatogram on the left panel illustrates a typical SEC purification. Elution fractions were run on the SDS-PAGE (Right Panel). Lane 1 of each SDS-PAGE is concentrated NiNTA elution fractions prior to the chromatographic run. Lane two of each (ACP₁, ACP₂, and ACP₃) SDS-PAGE are the SEC elution fractions containing the target protein. Calculated yields were 15, 24, and 18 mg/L, for ACP₁, ACP₂, and ACP₃, respectively.

FIG. 8. Estimation of molecular weights of tandem ACPs. SEC fractions collected during purification were re-injected into the SEC column using a low-salt buffer (20 mM NaH₂PO₄, 50 mM NaCl, pH 7.8). Retention times indicated molecular weights of 30.0 kDa for ACP₁, 95.3 kDa for ACP₂, and 261.6 kDa for ACP₃. Lane one in each SDS-PAGE (Right Panel) corresponds to fractions eluted during the purification step, and lane 2 of each SDS-PAGE corresponds to samples after second chromatographic run using low-salt buffer.

FIG. 9. Thermal unfolding monitored by circular dichroism. Thermal unfolding was monitored at 222 nm in a temperature range of 20-95° C. No significant differences in thermal unfolding were present among samples. Average Tm for all samples was 40.9° C.±1.8° C. After the final temperature of 95° C., there was a visible precipitate for all samples.

FIG. 10. Far-UV CD scans of thermal unfolding. Far-UV CD scans were collected at different temperatures (20-96° C.) in the range of 190-250 nm. A) ACP₁, B) ACP₂, C) ACP₃. Consistent with the melting curves monitored at 222 nm, we observed a loss of ellipticity in the regions associated with α-helical structure, specifically at the 222, 208, and 195 nm bands. All three proteins displayed a notable loss in ellipticity when scans were performed at 40° C., substantiating an average Tm of 40.9° C. determined by the denaturation curves.

FIG. 11. Solution structures of tandem ACPs by SAXS. Top panels are A) SAXS/WAXS curves obtained from the scattering data, and B) P(r) functions. Bottom panels are structural models for C) ACP₁, D) ACP₂, and E) ACP₃. Models on the left side are structures generated from the experimental SAXS scattering data, and models on the right are three-dimensional models built on the I-Tasser platform. ACP domains are highlighted in blue, the amino acid linkers in green, and the His-tag in yellow.

FIG. 12. Fatty acid profiles and growth curves of E. coli cultures harboring tandem ACPs. Panels A, C, and E correspond to the fatty acid profiles, and the panels B, D. and F are the growth curves. A-B) 37° C. C-D) 22° C., and E-F) 15° C. At every temperature, 16:0 was the prevalent species, and tandem ACPs did not appear to affect the distribution of fatty acids or bacterial growth. Error bars are the SEM of biological triplicates.

### DETAILED DESCRIPTION

Unless defined otherwise, all technical and scientific terms used herein have the same meaning as is commonly understood by one of ordinary skill in the art. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.). Springer Verlag (1991); and Hale & Marham, The Harper Collins Dictionary of Biology (1991). These references are intended to be exemplary and illustrative and not limiting as to the source of information known to the worker of ordinary skill in this art. As used herein, the following terms have the meanings ascribed to them below, unless specified otherwise.

It is noted here that as used in this specification and the appended claims, the singular forms “a,” “an,” and “the” also include plural reference, unless the context clarity dictates otherwise.

As used herein, the terms “or” and “and/or” are utilized to describe multiple components in combination or exclusive of one another. For example, “x, y, and/or z” can refer to “x” alone, “y” alone. “z” alone. “x, y, and z,” “(x and y) or z,” “x or (y and z),” or “x or y or z.”

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As utilized in accordance with the present disclosure, the following terms, unless otherwise indicated, shall be understood to have the following meanings. Unless otherwise required by context, singular terms shall include pluralities and plural terms shall include the singular.

Fatty Acid (FA) biosynthesis is a ubiquitous pathway in almost all prokaryotic and eukaryotic organisms. FA biosynthesis in bacterial and microbial cultures provides a unique solution to sustainable generation of biofuels and biomaterials as well as human nutrition. Although FA biosynthesis can be carried out in a wide range of organisms, E. coli offers advantages of low environmental impact, short production times, and ease of genetic manipulation.

E. Coli FA biosynthesis takes place through action of a dissociated system in which several different enzymes catalyze a series of chemical steps for the condensation of acyl units. Each protein is encoded by a separate gene and as such, in E. coli, acyl carrier proteins (ACPs), are separate domains that act as independent entities. While E. coli is the preferred microorganism for FA biosynthesis, one skilled in the art will understand that the dissociated system is common among other bacteria, plants, and algae, such as, but not limited to Yarrowia Lipolitica (a yeast), Synechococcus elongatus (a cyanobacteria) and Thraustochytrids (marine protists). (Ohlrogge, J. B. & Jaworski, J. G. Regulation of fatty acid synthesis. Annu. Rev. Plant Physiol. Plant Mol. Biol. 48, 109-136 (1997); Rock, C. O. & Jackowski, S. Forty years of bacterial fatty acid synthesis. Biochem. Biophys. Res. Commun. 292, 1155-1166 (2002). As such, one skilled in the art will understand that the biosynthesis of FA described can also be achieved in a wide range of additional microorganisms, including but not limited to other bacteria, plants, and algae. One of skill in the art would further understand that a wide variety of organisms could be genetically modified with an artificially fused multi-ACP gene and obtain a higher yield of fatty acids in that organism. The term artificially linkage as used herein is synonymous with recombinant linkage, gene fusion, or genetically fused. All terms convey an ACP-linker interaction that has been modified via laboratory techniques.

Each FA elongation cycle consists of four reactions required to extend the length of the acyl chain by two carbons. Initiation of FA biosynthesis requires the carboxylation of acetyl-CoA to produce malonyl-CoA.

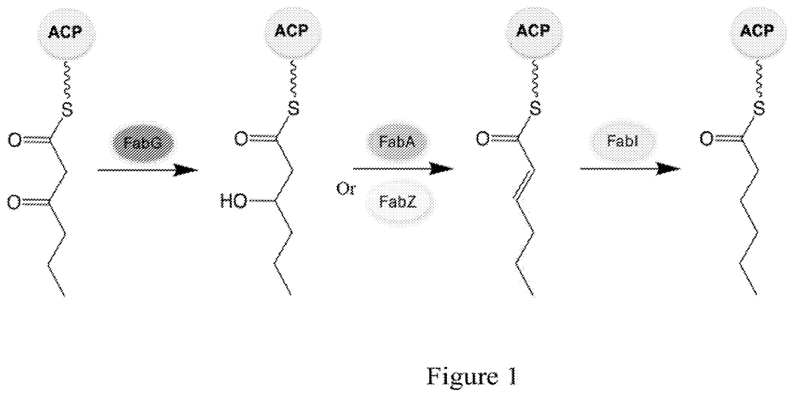

Continued FA biosynthesis requires concerted action of multiple enzymes that catalyze condensation of malonyl-CoA units, resulting in a fatty acyl chain that becomes modified by ketoreductase, dehydratase, and enoyl reductase enzymes. During biosynthesis of FAs, all starting materials and intermediates are covalently linked to the free thiol of the 4′-phosphopanthetheine prosthetic group in the ACP domain which serves as a shuttle from one active site to the next (FIG. 1). ACPs are also important and serve homologous functions in other metabolic pathways such as the biosynthesis of polyketides and non-ribosomal peptides by enzyme systems that employ a molecular logic similar to that of fatty acid synthases. While ACP functions as an independent protein in many organisms, there are species that contain covalently linked ACP domains in tandem to carry out their function.

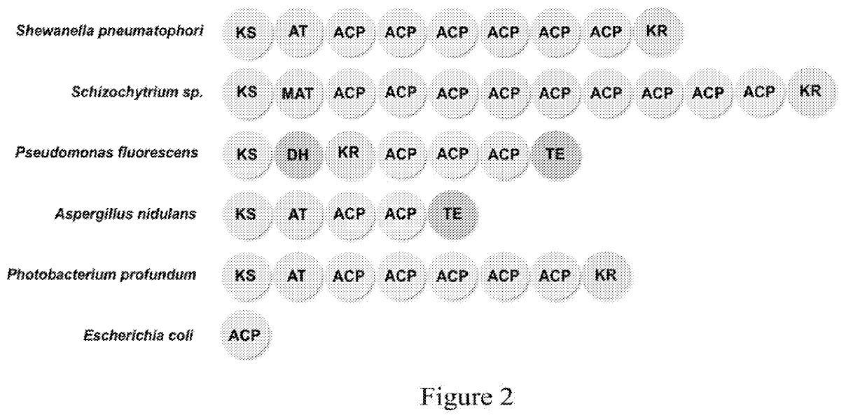

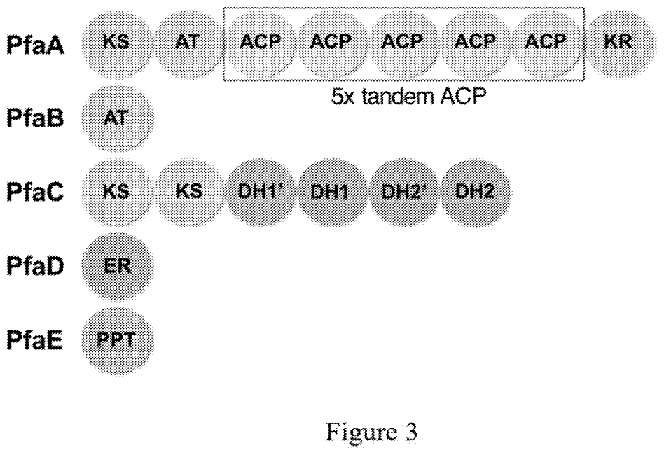

The presence of tandem ACP domains is present in synthase systems involved in production of several vital products, for example, the polyunsaturated fatty acid (PUFA) synthases, and polyketide synthases (PKSs). Many species of PUFA producing marine bacteria contain between 2-9 tandem (FIG. 2) ACP domains in their PUFA synthases, as seen in Photobacterium profundum synthase which contains 5 covalently linked ACP domains (FIG. 3). Examples of PKSs with tandem ACP domains have also been identified in numerous organisms including Pseudomonas fluorescens, although most known PKSs contain a single ACP domain for each cycle of chain elongation.

The advantages of tandem ACP arrangement in synthase systems have been reported for both biosynthesis of FAs and polyketides. The effect of tandem ACP domains is proportional increases in PUFA yields to the number of functional ACPs. Further, ACP domains are functionally equivalent regardless of their physical location. The structure and number of ACP domains control PUFA productivity as revealed by the by increased PUFA yields with the insertion of either inactivated or overly active ACP domains. The PfaA subunits which harbor ACP domains and their linkers are exchangeable between organisms and can influence the FA yields in a number of organisms. In addition, the production of polyketide antibiotics also benefits from the presence of tandem ACPs. Previous work by the inventors elucidated the solution structure of tandem ACPs from P. profundum, demonstrating that they adapt an elongated beads-on-a-string arrangement with flexible linkers and relatively independent domains. Elongated beads on a string, as used herein, refers to an arrangement in which multiple domains are linked together in series using a peptide linker with no contact or interaction between the domains. The arrangement is flexible and hypothesized not to be rigid as evidenced by the SAXS-derived structures. The domains do not interact with one another as evidenced by the highly similar unfolding temperatures of ACP1, ACP2 and ACP3.

In E. coli. ACP is an acidic, 78 amino acid protein, encoded by the acpP gene. ACP importance is reflected in its abundance, composing 0.25% of all soluble proteins in E. coli. ACP-bound intermediates are used in the biosynthetic pathway, while degradation is specific towards CoA-acyl chains. Thus, aside from its role as a shuttle for fatty acyl intermediates, ACP also plays a part in the regulation of the anabolic/catabolic pathways.

Given the importance of ACP in fatty acid production, a common strategy to enhance fatty acid yields is to overexpress ACP. However, this strategy is unviable as attempts to overexpress recombinant ACP in E. coli result in impaired cell growth, primarily by inhibition of glycerol-3-phosphate acyl transferase.

Interestingly, polyunsaturated fatty acid (PUFA)-producing bacteria are known to enhance their fatty acid production by harboring multiple ACP copies in tandem arrangements. For instance the expression of ACP from Azospirillum brasilense in E. coli increased the production of octadecenoic acid (18:1) 2-fold, demonstrating the validity of a fatty acid enhancement strategy based on the optimization of ACP. Further, fatty acid production yield in Shewanella japonica and in other organisms is proportional to the number of active ACP domains in the tandem arrangement. Conversely, the elimination of domains from the tandem arrangement results in a decrease in fatty acid production.

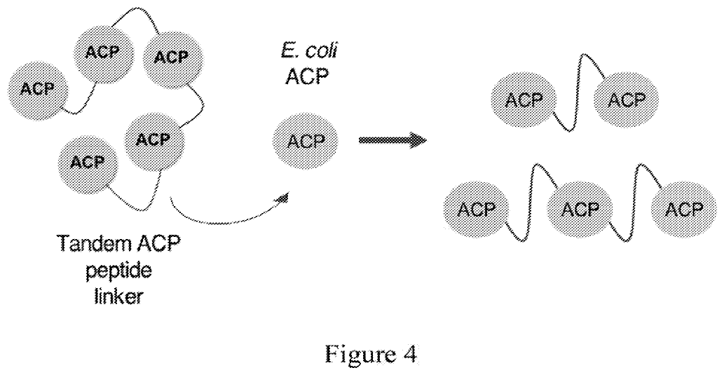

The current disclosure describes artificially linked ACPs as a means to optimize fatty acid production. The ACPs are arranged in an acpP-linker (or linker-acpP) intermediate prior to linkage of ACPs via peptide linkers. This is advantageous over current methods that increase production of native E. coli ACP domains which is toxic to the cell. Consistent with this, herein we describe ACP domains from E. coli artificially linked using the naturally occurring linker PfaA from P. profundum (FIG. 4). By mimicking the tandem ACP arrangement seen in PUFA and polyketide synthases fatty acid yields were increased with this fused tandemization.

Thus, in one embodiment the disclosure describes a heterodimer protein, comprising acyl carrier proteins (ACPs), comprising a population of ACPs and a population of linkers, wherein the population of ACPs are artificially covalently linked via the population of peptide linkers.

In one aspect the population of ACP artificially linked via the population of peptide linkers is a linked tandem ACP. The peptide linker can link a population of ACPs via covalent or non-covalent bonding. In a preferred embodiment, the peptide linker is a fragment from the pfaA multienzyme protein from Photobacterium profundum. The FAS peptide linker can also be derived from organisms, including but not limited a wide range of additional microorganisms, including but not limited to other bacteria, plants, and algae. One of skill in the art will know this further includes, but is not limited to, Escherichia coli (bacteria), Yarrowia Lipolitica (yeast), Synechococcus elongatus (cyanobacteria) and Thraustochytrids (marine protists).

A preferred peptide linker has a polypeptide sequence according to SEQ ID NO. 7, or a polypeptide with at least 75% identity to SEQ ID NO.7, homologs or functionally conserved variants thereof.

In one aspect of the current disclosure the artificially covalently liked ACP peptide has an arrangement wherein the carboxy terminus of the APC is covalently linked to the amino terminus of the peptide linker and wherein the carboxy terminus of the linker is covalently linked to the amino terminus of a second APC. In a further aspect, the artificially covalently linked peptide arrangement is repeated. In a preferred aspect the linked ACPs are arranged in a multi-domain arrangement. In yet another aspect of the preferred arrangement the multi-domain arrangement is a flexible multi-domain arrangement and characteristic of a beads-on-a string arrangement. One skilled in the art will recognize that artificial linkage of the ACP via, peptide linkers can result in additional arrangements including a branched or tree-like arrangement wherein ACPs are linked through cysteine residues. Further, nanoparticles, decorated with multiple ACPs could also be used to link the ACP residues. One skilled in the art would recognize that additional arrangements may require the use of an enzyme system reconstituted in vitro using purified enzyme components.

Historically, production of fatty acids using E. coli has been characterized by low activity that results in low yields. Further attempts to increase fatty acid production to-date have largely failed because increasing production of ACP domains in E. coli is toxic to the host related in-part to inhibition of key intermediates in the fatty acid biosynthetic pathway including glycerol-3-phosphate acyl transferase. In contrast, the current disclosure discloses advantageous, artificially linked ACPs wherein fatty biosynthesis is increased without toxicity to the host. Consistent with this, in another embodiment fatty acid biosynthesis production of tandem linked ACP is increased versus a single-domain ACP.

Consistent with this, the current disclosure provides methods for making artificially linked ACPs. In the preferred method artificially linked ACPs are made by constructing artificially linked multi domain ACP fragments; cloning and expressing the artificially linked multi domain ACP fragments to create artificially linked multi domain ACP proteins; purifying a first plurality of artificially linked multi domain ACPs and quantifying fatty acid yield of a second plurality of artificially linked multi domain ACPs. Multi-domain arrangements as used herein refers to a series of independently folded protein regions, also called domains, that are linked together covalently. A multi-domain arrangement could contain several copies of the same domains linked together or entirely different domains linked together. In this case, the covalent linkage or attachment is provided by a short peptide linker, but covalent linkage could be achieved through a number of different chemical methods such as bi-functional or multi-functional cross-linking agents. Although the multi-domain arrangement described herein is in linear or series, other multi-domain arrangements could be branched (e.g. tree like) or dendrimeric.

In one aspect a population of ACPs is linked via a population of peptide linkers to form an artificially linked ACP. In another aspect the artificially linked ACPs are linked in tandem. The peptide linker can link a population of ACPs via covalent or non-covalent bonding. In a preferred embodiment, the peptide linker is a fragment from the pfaA multienzyme protein from Photobacterium profundum. The PUFAS peptide linker can also be derived from organisms, including but not limited a wide range of additional microorganisms, including but not limited to other bacteria, plants, and algae. One of skill in the art will know this further includes, but is not limited to, Escherichia coli (bacteria), Yarrowia Lipolitica (a yeast), Synechococcus elongatus (a cyanobacteria) and Thraustochytrids (marine protists).

A preferred peptide linker has a polypeptide sequence according to SEQ ID NO. 7, or a polypeptide with at least 75% identity to SEQ ID NO.7, homologs or functionally conserved variants thereof.

More specifically, in the preferred method the artificially linked ACP fragments are arranged such that the carboxy terminus of the APC covalently linked to the amino terminus of the peptide linker and wherein the carboxy terminus of the linker is covalently linked to the amino terminus of a second APC. In one aspect the artificially covalently linked peptide has a repeated ACP-linker-ACP arrangement as described.

In the preferred method the multi-domain ACP fragments are tandemly linked and comprised of 2 or 3 ACP units linked by a peptide linker (ACP2 and ACP3 respectively). However, one of ordinary skill in the art will recognize that any number of ACP units can be linked resulting in a number of configurations.

In another aspect of the preferred embodiment, the multi domain ACP fragments are cloned and expressed in a host. In a preferred embodiment the host is an E. coli cell, however one of ordinary skill in the art will recognize that a number of host cells can be used for cloning and expression. In a preferred aspect of the method, host cells are grown at 37° C. until the desired optical density is achieved. In one aspect of the preferred method the desired optical density is 0.2 to 0.3, however one of skill in the art will recognize a range of optical densities can be used. After reaching the desired optical density protein expression is induced cells harvested, and a first plurality of artificially linked multi domain ACPs harvested for downstream analysis.

In one aspect downstream analysis is performed to determine molecular weight, in another aspect the analysis is used to determine structure and three dimensional models of the proteins. In yet another aspect of the method thermal characteristics of artificially linked multi domain ACPs are determined. More particularly, in the preferred method thermal denaturation data and small angle X-Ray are used to determine the structure of artificially linked multi-domain ACPs. In a preferred aspect of the method, the proteins display an elongated arrangement of quasi-independent domains, similar to beads-on-a-string with little or no contact between domains. In further aspect of the method the beads-on-a-string orientation can be verified by obtaining a pair distribution function (P(r)) of the artificially linked multi-domain ACP constructs. However, one of skill in the art will recognize that the artificially fused tandem ACPs may take on alternate structures as well. These may include branched or tree-like arrangements wherein ACPs are linked through cysteine residues. Further, nanoparticles, decorated with multiple ACPs could also be used to link the ACP residues. One skilled in the art would recognize that additional arrangements may require the use of an enzyme system reconstituted in vitro using purified enzyme components.

In a further preferred aspect of the method, the multi domain ACP fragments are cloned and expressed in a host. In a preferred embodiment the host is an E. coli cell, however one of ordinary skill in the art will recognize that a number of host cells can be used for cloning and expression. The host cells can be grown at a wide range of temperatures and fatty acid production quantified. However, in a preferred aspect of the method, host cells are grown at 37° C., 22° C., and 15° C., cells harvested and fatty acid production quantified.

### EXAMPLES

The Examples that follow are illustrative of specific embodiments of the disclosure, and various uses thereof. They are set forth for explanatory purposes only and should not be construed as limiting the scope of the disclosure in any way.

### Materials Used in Examples

Solutions and equipment used in the following Examples are briefly provided herein.

Amplification of ACPs and linkers was performed using a Thermal Cycler from Eppendorf and the PCR products were separated by agarose gel electrophoresis and purified using the QIAQuick Gel Extraction Kit (Qiagen). Primers were chosen using the Thermo Scientific Multiple Primer Analyzer tool. The PCR fragments were cloned into the pET-200/TOPO (Invitrogen), and E. coli strain TOP10 (Invitrogen).

Protein Expression was performed on E. coli BL2I(DE3)—Codon Plus RIL cells (Invitrogen), liquid Luria-Bertani medium supplemented with 0.4% glycerol, 1% glucose, and containing kanamycin (100 mg/L) and chloramphenicol (25 mg/L). Protein expression was induced with 1 mM IPTG and cells harvested by centrifugation on a Sorvall Lynx 4000 Centrifuge using a Fiberlite™ F14-14×50cy Fixed-Angle Rotor (Thermo). Lysis buffer was used to re-suspend pellets (20 mM Tris, 500 mM NaCl, 1 mM DTT, 20% glycerol, pH 7.8) in presence of lysozyme (10 mg/ml), DNase (1 mg/ml), 2X protease inhibitor cocktail (Pierce). Soluble lysates were filtered through a column filled with Ni-Sepharose (Sigma) previously equilibrated with buffer+5 mM imidazole and washed twice with the same buffer+10 mM imidazole. His-tagged proteins were eluted in the in the corresponding buffer containing 200 mM imidazole. Purities of elution fractions were analyzed by SDS-PAGE. Protein yields were calculated using Nanodrop A280 quantification and values obtained by dividing total milligrams of protein per volume of culture.

Thermal stability determination utilized NiNTA elution fractions purified by size exclusion chromatography and exchanged into 20 mM NaH₂PO₄, 50 mM NaCl, pH 7.8. The signal was read in a Jasco 1 mm quartz cuvette on a Jasco-1500 spectropolarimeter.

Quaternary structures were analyzed using purified proteins infused into a Superdex 200 Increase 10/300 GL column (GE Healthcare) equilibrated in the NiNTA lysis buffers minus glycerol. Each run with the enzymes was preceded by a run with a mixture of standard proteins (GE Healthcare) [aprotinin (6,500 Da), ribonuclease (13,700 Da), ovalbumin (44,000 Da), conalbumin (75,000 Da), aldolase (158,000 Da), ferritin (440,000 Da)] to generate a Kav vs. logMW curve. Elution volumes were determined using the Unicorn software integration function.

Solution structure determination utilized protein exchange into a 20 mM NaH₂PO₄, 50 mM NaCl, pH 7.8 and concentrated between 1-3 mg/ml. X-ray scattering experiments were conducted at the LiX beamline at the National Synchrotron Light Source II at Brookhaven National Laboratory, Brookhaven, N.Y. Data processing was performed using an automated Python-based package developed at LiX. The resulting data was combined and merged scattering pattern was analyzed using PRIMUS. DAMMIN, and DAMMIF of the ATSAS software package and the Rg obtained through the indirect transform algorithm in GNOM. (Franke, D., et al. ATSAS 2.8: a comprehensive data analysis suite for small-angle scattering from macromolecular solutions. J. Appl. Crystallogr. 50, 1212-1225 (2017)).

Extraction and modification of fatty acids required cultures grown in triplicate in liquid LB media in the absence and presence of 1 mM IPTG. Dried pellets were dissolved in NaCl, chloroform, MeOH, and vortexed. The organic phase was isolated after centrifugation and concentrated under N2. Finally, the concentrated fatty acids were re-suspended in a mixture of chloroform:MeOH (2:1). A chloroform:MeOH mixture was used to convert fatty acids to their respective methyl esters. Methyl heneicosanoate was added as an internal standard prior to the addition of methanolic HCl. Fatty acids were isolated by extraction with hexane, dried under N2, and resuspended in 200 μL of hexane.

Fatty acid profiling and quantification was performed using a GC/MS-QP2010 (Shimadzu) with a fused-silica FAMEWAX capillary column (0.25 mm×30 m) (Restek). Mass Spec data was processed using the GC/MS Solution Post run Analysis software (Shimadzu) for metabolite identification using the NIST 2011 spectral mass library. Data was analyzed using GraphPad Prism 6.

### Example 1. Primer Design of acpP

DNA sequence for E. coli DH10B acpP (Ref. sequence NC_000913.3:11516151151851) was obtained from NCBI. The linker region corresponding to amino acids 1511-1538 of the pfaA gene from P. profundum (Accession No. CAG19871.1) were also used. The primers for the amplification of acpP contained overhangs of additional sequence that are complementary to either the 5′ or 3′ terminus of the linker sequence (Table 1; overhang shown in bold).

TABLE 1
Primers for the amplification of acpP

| SEQ | | |
|---|---|---|
| ID | Primer | |
| NO: | Name | Sequence (5′ → 3′) |
| 1 | acpP Fwd | CACCATGAGCACTATCGAAGAACGC |
| | TOPO | |
| 2 | acpP Rv + | TACATTTTGTGTAGACGCCTGGTGGCCGTTG |
| | Overhang | |
| 3 | acpP Fwd + | AAGTTCAAGGCACAATGAGCACTATCGAAGAACGC |
| | Overhang | |
| 4 | acpP Rv + | TTACGCCTGGTGGCCGTTG |
| | Stop | |
| 5 | pfaA Fwd | TCTACACAAAATGTAGCGATTC |
| 6 | pfaA Rv | TGTGCCTTGAACTTGTGCTG |

The reverse primers for the amplification of acpP contained overhangs complementary the 5′ terminus of the linker sequence, whereas forward primers contained overhangs complementary to the 3′ terminus of the linker sequence. The same principle applied for the amplification of the pfaA linker. All primers were purchased from the RCMI Core Lab at UPR Medical Sciences Campus after verification of secondary structure and primer dimer formation using the online Thermo Scientific Multiple Primer Analyzer tool.

### Example 2. Construction of Artificial Genes and Cloning

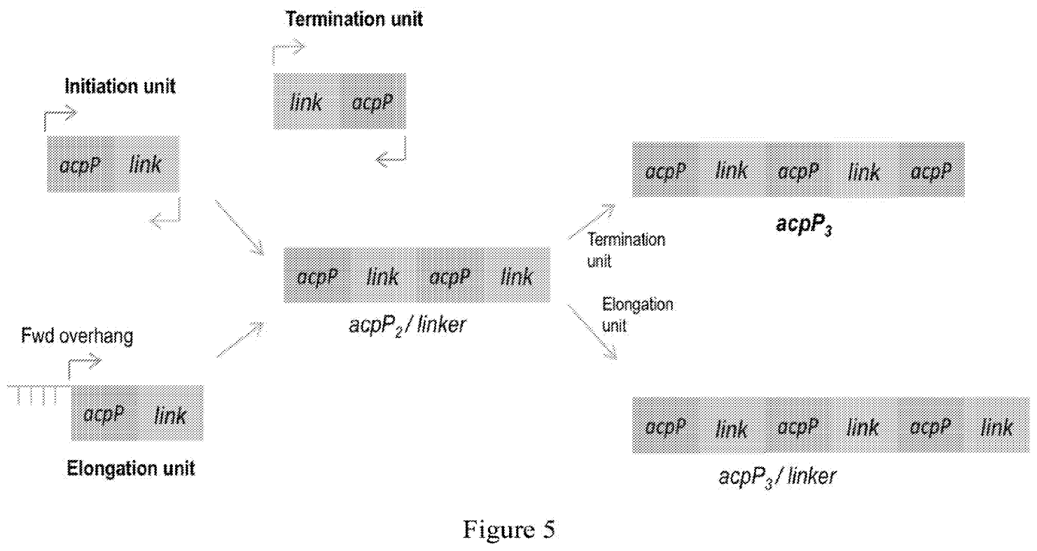

A general scheme for generation of fused tandem acpP by overlap PCR is presented in FIG. 5. For the amplification of acpP, approximately 2 ng of genomic DNA from E. coli DH10B cells was mixed with primers 1 and 4, using the same PCR parameters as for the tandem constructs.

Genes amplified using the overhanging forward primer contain an additional sequence complementary to the 3′ end of the linker gene. Genes amplified using the overhanging reverse primer contain additional sequence complementary to the 5′ end of the linker gene. Annealing of both products in PCR forms an acpP-linker (or linker-acpP) intermediate. Three basic building blocks were constructed to either extend or terminate each tandem gene: an initiation unit, an elongation unit, and a termination unit. The initiation unit contains a 5′CACC overhang sequence to facilitate the cloning into pET200/D-TOPO. Intermediates could either be extended by a subsequent PCR reaction with an elongation unit or prepared for cloning using a termination unit. The terminator unit contains a 3′TTA sequence encoding for a stop codon.

The sequential PCR reactions needed were performed as outlined in Table 2

PCR reactions were run for 30 cycles without primers, followed by 15 additional cycles with primers. Individual PCR reaction products were separated by electrophoresis on an agarose gel (2%) and purified using the QIAQuick Gel Extraction Kit (Qiagen).

All PCR reactions were preceded by a denaturation step at 95° C. for two minutes and finished with a final extension step at 68° C. for 3 minutes. PCR cycles included DNA denatured at 95° C. for 1 minute, followed by annealing at 45.8° C. for 1 minute and finished with an extension 68° C. for 1 minute.

The pfaA linker adds 84 base pairs to each construct. These 84 base pairs correspond to a 28 amino acid sequence of STQNVAIQTAAPVASASNGLDAAQVQGT (SEQ ID NO. 7). All constructs were cloned into pET-200/TOPO (Invitrogen) plasmids, transformed into E. coli strain TOP10 (Invitrogen), and their sequences were fully verified using the Sanger method

### 2.3.2. Construction of ACP Multimers and Molecular Cloning

TABLE 2
Parameters and steps for the construction of acpP2 and acpP3.

| | | |
|---|---|---|
| Product | Reaction I | Reaction II |

| | | | | | | |
|---|---|---|---|---|---|---|
| Initiation | Templates(s) | Fwd. | Rv. | Template (s) | Fwd. | Rv |
| unit | DH10B genome | 1 | 2 | [symbol] | 1 | 8 |

| | |
|---|---|
| PCR Parameter | PCR Parameters |

| | | | | | |
|---|---|---|---|---|---|
| Step | ° C. | Time | Step | ° C. | Time |
| Denaturation | [symbol] | 2:00 | Denaturation | [symbol] | 2:00 |
| Annealing | [symbol] | 1:00 | Annealing | [symbol] | 1:00 |
| Extension | [symbol] | 1:00 | Extension | [symbol] | 1:00 |

| | | |
|---|---|---|
| Product | Reaction I | Reaction II |

| | | | | | | |
|---|---|---|---|---|---|---|
| Elongation | Templates(s) | Fwd. | Rv. | Template (s) | Fwd. | Rv. |
| unit | DH10B genome | 3 | 2 | [symbol] | 3 | 8 |

| | |
|---|---|
| PCR Parameter | PCR Parameters |

| | | | | | |
|---|---|---|---|---|---|
| Step | ° C. | Time | Step | ° C. | Time |
| Denaturation | [symbol] | 2:00 | Denaturation | [symbol] | 2:00 |
| Annealing | [symbol] | 1:00 | Annealing | [symbol] | 1:00 |
| Extension | [symbol] | 1:00 | Extension | [symbol] | 1:00 |

| | | |
|---|---|---|
| Product | Reaction I | Reaction II |

| | | | | | | |
|---|---|---|---|---|---|---|
| Termination | Templates(s) | Fwd. | Rv. | Template (s) | Fwd. | Rv. |
| unit | DH10B genome | 3 | 4 | [symbol] | 5 | 4 |

| | |
|---|---|
| PCR Parameter | PCR Parameters |

| | | | | | |
|---|---|---|---|---|---|
| Step | ° C. | Time | Step | ° C. | Time |
| Denaturation | [symbol] | 2:00 | Denaturation | [symbol] | 2:00 |
| Annealing | [symbol] | 1:00 | Annealing | [symbol] | 1:00 |
| Extension | [symbol] | 1:00 | Extension | [symbol] | 1:00 |

| acpP₂ | | | acpP₃ | | | | | |
|---|---|---|---|---|---|---|---|---|
| Reaction I | | | Reaction I | | | Reaction II | | |
| Templates(s) | Fwd. | Rv. | Templates (s) | Fwd. | Rv. | Templates (s) | Fwd. | Rv. |
| [symbol] | [symbol] | [symbol] | [symbol] | [symbol] | [symbol] | [symbol] | [symbol] | [symbol] |
| PCR Parameters | | | PCR Parameters | | | PCR Parameters | | |
| Step | ° C. | Time | Step | ° C. | Time | Step | ° C. | Time |
| Denaturation | [symbol] | [symbol] :00 | Denaturation | [symbol] | [symbol] :00 | Denaturation | [symbol] | [symbol] :00 |
| Annealing | [symbol] | [symbol] :00 | Annealing | [symbol] | [symbol] :00 | Annealing | [symbol] | [symbol] :00 |
| Extension | [symbol] | [symbol] :00 | Extension | [symbol] | [symbol] :00 | Extension | [symbol] | [symbol] :00 |

[symbol]

indicates data missing or illegible when filed

### Example 3. Protein Expression and Purification

E. coli BL21(DE3)-Codon Plus RIL cells (Invitrogen) were transformed with each plasmid and grown in liquid Luria-Bertani (LB) medium supplemented with 0.4% glycerol, 1% glucose, kanamycin (100 m/L), and chloramphenicol (25 mg/L). Cultures were grown at 37° C., 250 rpm shaking speed until OD600=0.2.−0.3, at which point the temperature was lowered to 22° C. Once the OD600=0.5-0.6 protein expression was induced with 1 mM IPTG. After four hours cell pellets were harvested by centrifugation at 4° C. and 11,000×g on a Sorvall Lynx 4000 Centrifuge using a Fiberlite™ F14-14×50cy Fixed-Angle Rotor (Thermo). Cell pellets were stored at −20° C. overnight and re-suspended in lysis buffer (20 mM Tris, 500 mM NaCl, 1 mM DTT, 20% glycerol, pH 7.8) in the presence of lysozyme (10 mg/mi). DNAse (1 mg/ml), 2X protease inhibitor cocktail (Pierce) and sonicated. Lysates were collected by centrifugation (11,000×g, 4° C., 30 min). The soluble lysates were poured through a column filled with Ni-Sepharose (Sigma) that had been equilibrated with the corresponding higher salt buffer noted above +5 mM imidazole and washed twice with the same buffer+10 mM imidazole. His-tagged protein fractions were eluted in the in the corresponding buffer containing 200 mM imidazole. Fractions were infused into a size exclusion chromatography Superdex 200 Increase 10/300 GL column (GE Healthcare), equilibrated in the NiNTA lysis buffer minus glycerol, and eluted at a flowrate of 0.8 mL/min. Purities of elution fractions were analyzed by SDS-PAGE and protein yields calculated using Nanodrop A280 quantification by dividing total milligrams of protein per volume of culture.

### Results

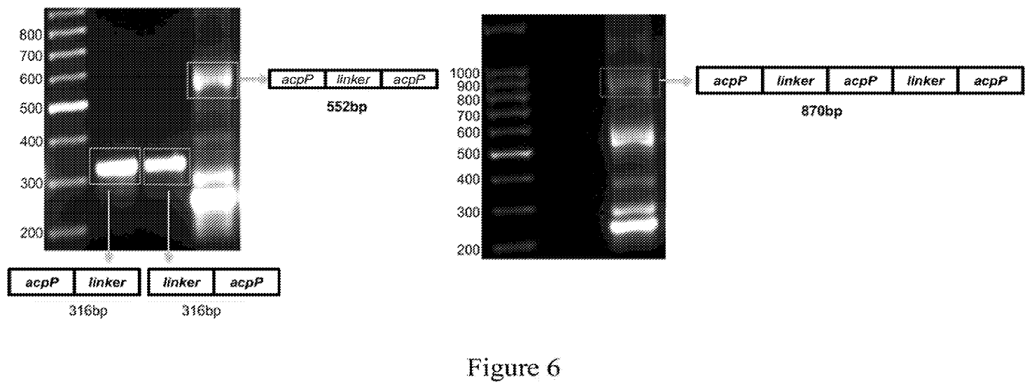

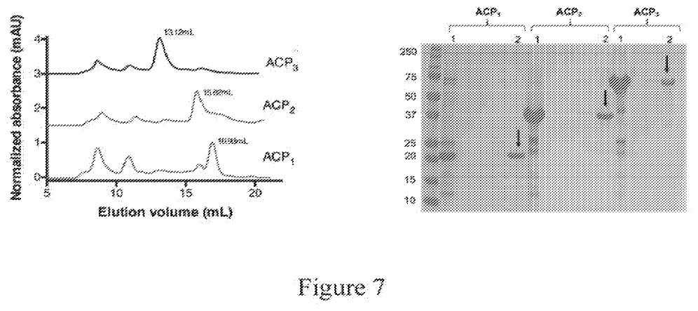

Two tandem acpP genes corresponding to a dimer and a trimer were constructed and named acpP₂ and acpP₃, respectively. Genetic constructs were cloned into pET-200/D-TOPO (FIG. 6), and their protein products expressed in E. coli cultures at 22° C. as evidenced by SDS-PAGE analysis. Plasmids were sent for sequencing and verified by the Sanger method. As shown in FIG. 6 artificial genes were visualized on a 2% agarose gel. A band in the 552 bp region was observed corresponding to acpP₂. The 552 base pairs are accounted for by the base pairs corresponding to the linker (84 bp) plus the base pairs corresponding to two acpP sequences (234 bp each). A band in the 870 bp region is consistent with acpP₃. Both bands (acpP₂ and acpP₃) were purified by gel extraction and cloned into pET200/D-TOPO vectors. The purification scheme used resulted in highly pure elution fractions with no indication of His-tag sequestering inside the hydrophobic core (FIG. 7). Yields were calculated to be 15, 24, and 18 mg/L, for ACP1, ACP2, and ACP3, respectively. The sequences of the ACP constructs are shown in Table 3.

TABLE 3
Sequences of the ACP Constructs

| SEQ | | |
|---|---|---|
| ID | | |
| NO. | Gene | Sequence |
| 7 | Photobacterim | STQNVAIQTAAPVASASNGLDAAQVQGT |
| | Profundum Fatty | |
| | Acid Synthetase | |
| 8 | E. coli Acyl | MAILGLGTDIVEIARIEAVIVRSGERLARR |
| | Carrier | VLSDNEWEIWKTHHQPVRFLAKRFAVKEAA |
| | Protein (ACP) | AKAFGTGIRNGLAFNQFEVFNDELGKPRLR |
| | | LWGEALKLAEKLGVANMHVTLADERHYACA |
| | | TVIIES |

### Example 4. Determination of Molecular Weights by Size Exclusion Chromatography

Purified proteins were exchanged into a low-salt system (20 mM NaH₂PO₄, 50 mM NaCl, pH 7.8) and proteins re-eluted in the in the corresponding buffer containing 200 mM imidazole. Purities of elution fractions were analyzed by SDS-PAGE. Each run with the proteins was preceded by a run with a mixture of standard proteins (GE Healthcare) [aprotinin (6,500 Da), ribonuclease (13,700 Da), ovalbumin (44,000 Da), conalbumin (75,000 Da), aldolase (158,000 Da), ferritin (440,000 Da)] to generate a Kav vs. logMW curve, where Kav=(Ve-Vo)/(Vc-Vo), Ve is the elution volume, Vc is the total column volume, and Vo is the column void volume. Proteins were eluted at a flowrate of 0.8 mL/min, and the elution volumes were determined using the Unicorn software integration function. The resulting standard curve was used to estimate the molecular weight of the proteins of interest.

### Results

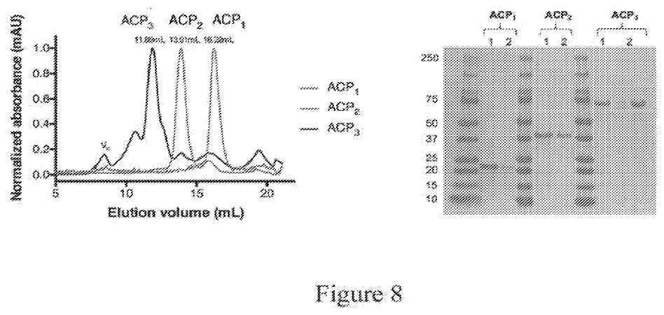

Using size exclusion chromatography (SEC), the molecular weights of each construct were determined and compared with those of the theoretical products. The chromatogram of the SEC purification step displayed retention times corresponding to molecular weights of 21.1 kDa for ACP1, 36.7 kDa for ACP2, and 141.4 kDa for ACP3. The analytical runs using a lower salt buffer (20 mM NaH₂PO₄, 50 mM NaCl, pH 7.8) had chromatograms with retention times that showed an increase in apparent size (molecular weight): 30.0 kDa for ACP, 95.3 kDa for ACP2, and 261.5 kDa for ACP3 (FIG. 8). The increase in apparent molecular weights is not the result of intermolecular disulfide bonds forming due to the lack of DTT, since neither ACP nor the amino acid linker contain cysteine residues within their amino acid sequence. While the identity of the buffer clearly affects the apparent molecular weights of each protein, these molecular weights are consistently higher than what would be expected based on the amino acid sequence of these proteins, regardless of the buffer (Table 4). The size exclusion chromatography profiles for ACP₁, ACP₂ and ACP₃ all suggest a structure that is larger than would be expected by the sequence (141 kDa vs 35 kDa by sequence for ACP₃), which is consistent with an elongated arrangement of ACP domains. These observations on the possible elongated structure of fused multi-ACP domains that were derived from biophysical characterization methods were also confirmed by Small Angle X-Ray Scattering (SAXS).

TABLE 4
Expected vs. obtained molecular weights of tandem ACPs

| | | M.W determined | M.W determined |
|---|---|---|---|
| | Expected | by SEC (kDa) | by SEC (kDa) |
| | M.W | (20 mM Tris, | (20 mM NaH₂PO_{4,} |
| | (kDa) | 500 mM NaCl) | 50 mM NaCl) |
| ACP₁ | 12 | 21.1 | 30.0 |
| ACP₂ | 24 | 36.7 | 95.3 |
| ACP₃ | 37 | 141.4 | 261.5 |

This suggests that either the tandemized ACPs form higher oligomers, or form elongated structures

### Example 5. Thermal Stability Determination by Circular Dichroism

Proteins were exchanged into 20 mM NaH2PO4, 50 mM NaCl, pH 7.8, using size exclusion chromatography. All samples came from new aliquots that were not previously thawed and refrozen. The absence of DTT did not alter elution volumes. Protein fractions were diluted to 0.1 mg/mL and placed in a Jasco 1 mm quartz cuvette. No more than 20 minutes elapsed from the time the samples were collected from the Size Exclusion Chromatography (SEC) column to when the samples were placed in the circular dichroism (CD) cuvette. No further sample preparation was performed after dilution. Thermal denaturation was measured by monitoring the CD signal at 218 nm as a function of temperature in the range of 20-95° C. (1° C./min) in a Jasco-1500 spectropolarimeter using the Interval Temperature Scan Measurement program. The program was set to hold the temperature after every 10° C. and record far-UV CD spectra from 190-250 nm until the final temperature was reached. Far-UV spectra was reported as an average of three scans. Precipitation was observed at the end of the run in every sample. Parameters for Tm scan were: CD scale 200 mdeg/1.0dOD, D.I.T 1 s, bandwidth 1 nm. Parameters for CD spectra scans were: CD scale 200 mdeg/1.0dOD, D.I.T 4 s, bandwidth 1 nm, scan speed 50 nm/min. CD units (mdeg) were converted to θMRE units using the equation θMRE=(deg*MRW)/(10*c*b), where MRW is the mean residue weight (Da), c is the enzyme concentration (g/mL), and b is the cell path length (cm). Denaturation analysis was performed using the built-in Thermal Denaturation Analysis Program.

### Results

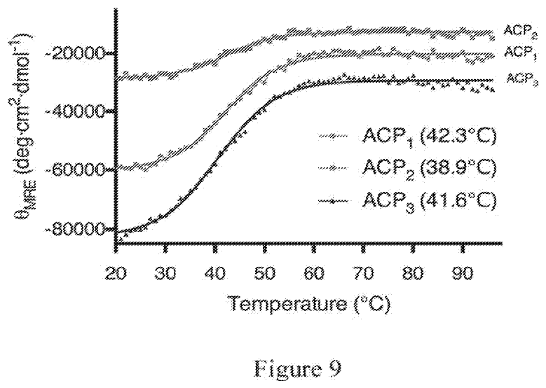

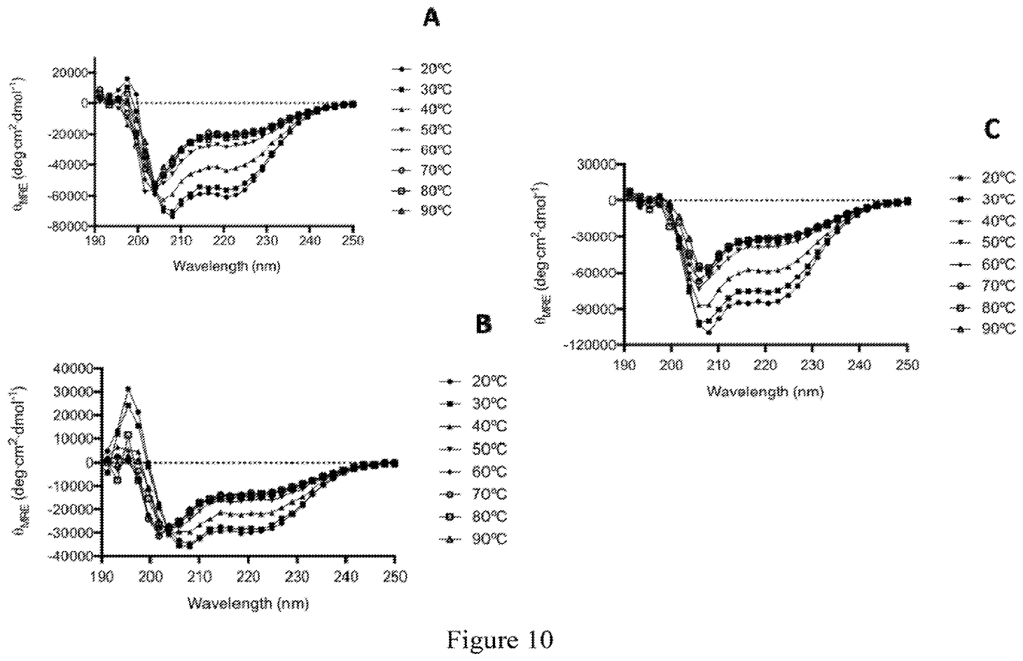

In order to determine whether the artificial tandemization of ACP stabilizes domains, we employed circular dichroism (CD) spectroscopy to assay thermal stabilities. We thus monitored thermal unfolding across a temperature gradient and measured the melting point (Tm) of each protein. We observed very little difference among samples, suggesting that ACP tandemization does not significantly impact stability. Tm values were 42.3, 38.9, and 41.6° C. for ACP1, ACP2, and ACP3, respectively (FIG. 9). This corresponds to a mean Tm of 40.9° C. and a standard deviation of 1.8° C. The melting curves were complemented with scans in the far-UV region. Scans performed in the 190-250 nm range also revealed the characteristic loss of ellipticity for α-helical structures at the 222, 208, and 195 nm bands (FIG. 10). Consistent with the melting curves, the decrease in ellipticity at these bands is first observed at 40° C. substantiating an average Tm of 40.9° C. determined by the denaturation curves. Unfolding was not fully reversible for any protein, as the CD signals did not return to their initial values when the temperature was lowered back to 20° C.

### Example 6. Solution Structures Using Small Angle X-Ray Scattering

To generate models for the solution structure of tandem ACPs, we employed Small Angle X-Ray Scattering (SAXS). Proteins were exchanged into 20 mM NaH₂PO₄, 50 mM NaCl, pH 7.8 and concentrated between 1-3 mg/ml. X-ray scattering was conducted at the LiX beamline at the National Synchrotron Light Source II at Brookhaven National Laboratory, Brookhaven. N.Y. Enzymes and buffer solutions continuously flowed through a capillary and were exposed to the X-ray beam for 5 s and measurements carried out in triplicate.

Data processing was performed using an automated Python-based package developed at LiX. Two-dimensional scattering patterns from protein solutions were first converted into one-dimensional scattering profiles. The SAXS/WAXS data were merged, averaged, and buffer-only background values subtracted to obtain relative scattering intensity (I) as a function of the momentum transfer vector, q (q=(4π sin θ)/λ), where λ is the beam wavelength, and θ is the scattering angle. The resulting combined and merged scattering pattern was analyzed using PRIMUS, DAMMIN, and DAMMIF of the ATSAS software package using a resolution range of q={0.01−0.32 A}. The radius of gyration (Rg) value derived from the Guinier analysis (5.060.1 nm) corresponded well to the Rg obtained through the indirect transform algorithm in GNOM.

### Results

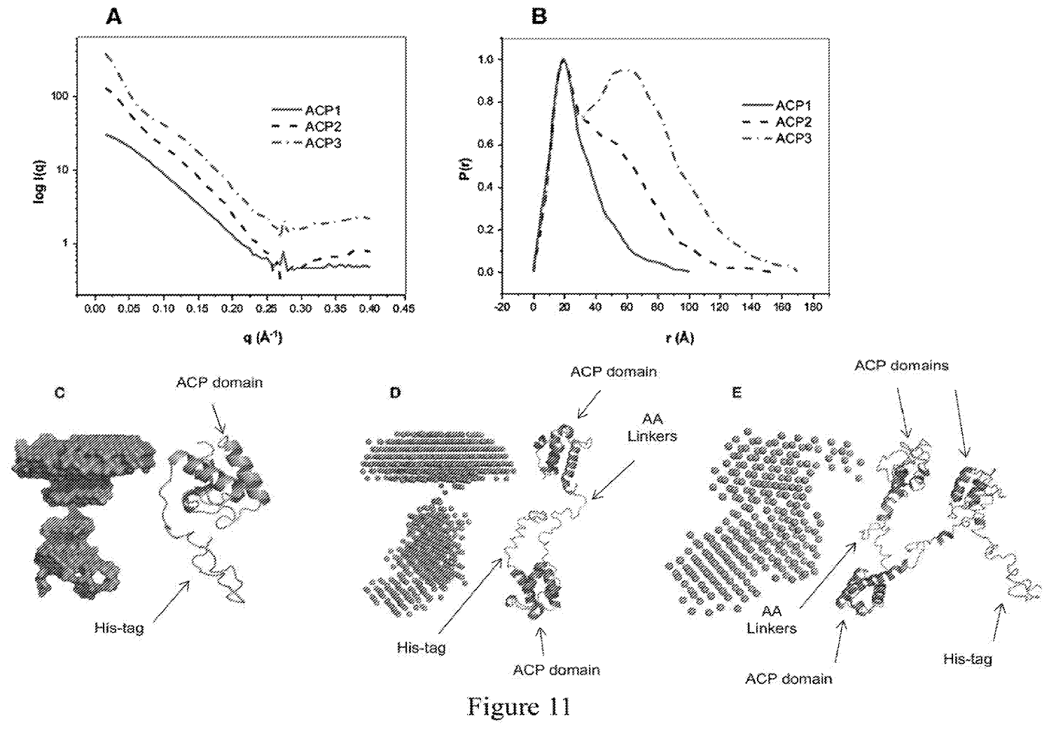

Analysis of the distance distribution functions (P(r)) revealed gyration radii of 23.5 Å for ACP1, 36.6 Å for ACP2, and 48.0 Å for ACP3. The maximum diameter of the proteins (Dmax) also increased as ACP domains were added, with Dmax values of 100 Å for ACP1, 155 Å for ACP2, and 170 Å for ACP3. The P(r) functions generated from the SAXS scattering data were consistent with dumbbell-like particles, and suggest that the artificial linkage of ACP results in the formation of beads-on-a-string arrangements. Additionally, the particle sizes of three-dimensional models generated on I-Tasser were able to accommodate all three tandem ACPs (FIG. 11). Further, the fact that the presence of additional ACP domains does not result in more stable structures is consistent with an elongated, beads-on-a-string arrangement.

### Example 7. Extraction, Modification and In Vivo Quantification of Fatty Acids

Cultures were grown in triplicate in LB media using the same procedure as before supplemented with 0.4% glycerol, 1% glucose, kanamycin (100 mg/L), and chloramphenicol (25 mg/L). Cultures were grown at 37° C., 22° C., and 15° C., in the absence and presence of 1 mM IPTG. After 27 hours, cells were harvested, frozen at −80° C., and lyophilized. Fatty acids were extracted using the Bligh-Dyer method (Bligh, E. G. & Dyer, W. J. A rapid method of total lipid extraction and purification. Can. J. Biochem. Physiol. 37, 911-917 (1959)). Briefly, dried pellets were dissolved in NaCl, chloroform, and MeOH and then vortexed. The organic phase containing fatty acids was isolated after centrifugation at 5000×g and concentrated under N2. Concentrated fatty acids were re-suspended in a mixture of chloroform:MeOH (2:1) to convert fatty acids to their respective methyl esters.

Extracted fatty acids in the chloroform:MeOH mixture were reacted with methanolic HCl and refluxed for 2 h under constant stirring and heating. Methyl heneicosanoate was added as an internal standard prior to the addition of methanolic HCl. Fatty acids were isolated by extraction with hexane, dried under N2, and resuspended in 200 μL of hexane.

Fatty acid profiling was performed using a GC/MS-QP2010 (Shimadzu) with a fused-silica FAMEWAX capillary column (0.25 mm×30 m) (Restek). The initial oven temperature was set to 130° C. for 5 min, followed by a temperature ramp from 130 to 250° C. at 4° C./min, and finally, held at 250° C. for 5 min, using helium as the carrier gas. Mass spectra data were obtained after electron impact ionization at 70 eV at an ion source temperature 200° C. in full scan mode between 50 and 600 amu. Temperatures of the injection port and the detector were set at 250° C. Data was processed with GC/MS Solution Postrun Analysis software (Shimadzu) for metabolite identification using the NIST 2011 spectral mass library.

Fatty acid yields were calculated as follows: First, the millimoles of each fatty acid were calculated using the formula mmol FA=(AFA/AIS)*CIS*Vchl, where (AFA/AIS) is the ratio of the areas of each fatty acid and the internal standard calculated from the gas chromatogram, CIS is the known concentration of the internal standard (M), and Vchl is the volume of chloroform:MeOH used to re-suspend the concentrated fatty acids (mL). Second, milligrams of each fatty acid per liter of culture was calculated as mg FA/L=(mmol FA*gcell*MW)/(gext*Vtot), where gcell is the total dry cell mass (g), gext is the dried cell mass used for the extraction (g), Vtot is the total culture volume (L), and MW is the molecular weight for each fatty acid (g/mol). Individual fatty acid yields were pooled in order to determine the amount of total fatty acids per liter of culture. Statistical analysis was performed using GraphPad Prism 6 and data presented as means±SEM. Associatedp-values were calculated using the Student's t-test, p<0.05.

### Results

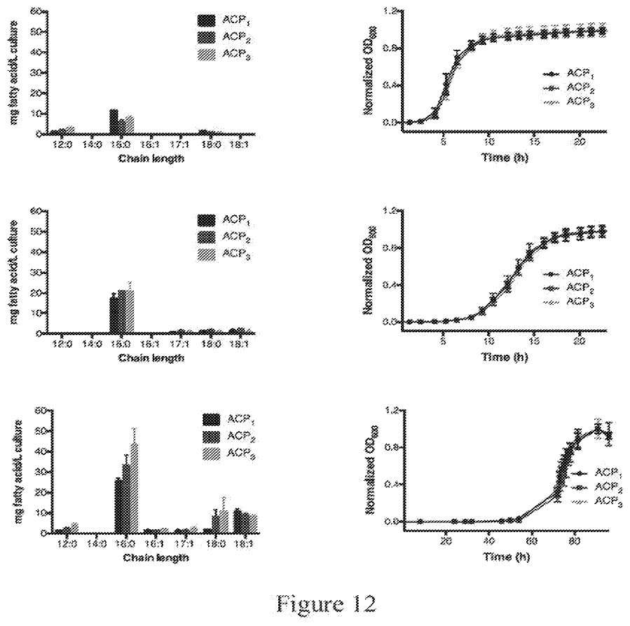

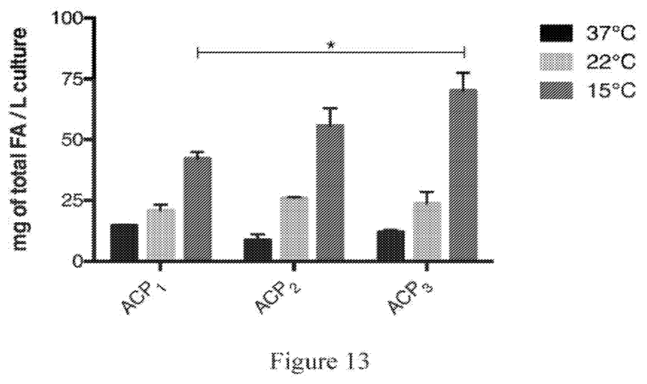

GC/MS analysis was used to quantify the yields of fatty acids produced in E. coli cultures harboring the different ACP constructs at three different temperatures. Overexpression of ACP in E. coli has previously been shown to inhibit cell growth because of the accumulation of unmodified apo-ACP. Test conditions were therefore carried out in the absence of IPTG in order to limit the amount of unmodified ACP in the medium. Consistent with previous studies, saturated fatty acids—mainly hexadecanoic acid (16:1)—were the prominent fatty acids in cultures grown at 37° C. (Ivancic, T., et al. Conditioning of the membrane fatty acid profile of Escherichia coli during periodic temperature cycling. Microbiology 155, 3461-3463 (2009); Feng, Y. & Cronan, J. E. Escherichia coli unsaturated fatty acid synthesis: Complex transcription of the fabA gene and in vivo identification of the essential reaction catalyzed by FabB. J. Biol. Chem. 284, 29526-29535 (2009); Marr, A. G. & Ingraham, J. L. Effect of temperature on the composition of fatty acids in Escherichia coli. J. Bacteriol. 84, 1260-1267 (1962); Neidleman, S. L. Effects of temperature on lipid unsaturation. Biotechnol. Genet. Eng. Rev. 5, 245-268 (1987)). No unsaturated fatty acids were detected at this temperature. At the lower temperatures, monounsaturated fatty acids, mainly cis-9-octadecenoic acid (18:1), are produced at significant levels, especially at 15° C. (FIG. 12). These results are summarized in Table 5.

TABLE 5
Fatty acid profiles of E. coli cultures harboring tandem ACPs

| | | | | | | |
|---|---|---|---|---|---|---|
| 12:0 | 14:0 | 16:0 | 16:1 | 17:1 | 18:0 | 18:1 |

Fatty acid profiles at 37° C. (mg/L)

| | | | | | | | |
|---|---|---|---|---|---|---|---|
| ACP₁ | 1.2 | — | 11.6 | — | — | 1.7 | — |
| ACP₂ | 1.8 | — | 5.9 | — | — | 1.1 | — |
| ACP₃ | 3.0 | — | 7.8 | — | — | 1.0 | — |

Fatty acid profiles at 22° C. (mg/L)

| | | | | | | | |
|---|---|---|---|---|---|---|---|
| ACP₁ | — | — | 17.4 | — | 0.6 | 1.5 | 1.4 |
| ACP₂ | — | — | 20.9 | — | 1.5 | 1.6 | 2.0 |
| ACP₃ | — | — | 20.1 | — | 0.7 | 1.2 | 0.9 |

Fatty acid profiles at 15° C. (mg/L)

| | | | | | | | |
|---|---|---|---|---|---|---|---|
| ACP₁ | 1.5 | — | 25.7 | 1.5 | 1.2 | 1.8 | 10.7 |
| ACP₂ | 2.5 | — | 33.2 | 1.6 | 1.3 | 8.0 | 9.2 |
| ACP₃ | 3.8 | — | 43.6 | 2.0 | 2.3 | 10.5 | 8.1 |

The cultures grown at 22° C. produced slightly higher overall yields than those grown at 37° C., however, this effect was consistent regardless of the number of ACP domains. The most significant difference was observed at 15° C., where each addition of an ACP domain increased the overall fatty acid yields. The fatty acid yield for ACP1 was 42.3±3.1 mg/L; the addition of ACP domains increased the yields to 55.8±8.9, and 70.2±9.1 mg/L, for ACP2 and ACP3, respectively. This corresponds to a 1.6-fold enhancement for ACP3 over control ACP1 (FIG. 13). The tandem ACPs did not impact the cell mass, as growth curves monitored for a set time period did not display significant differences (FIG. 12). Overall fatty acid yields are summarized in Table 6.

TABLE 6
Pooled Total fatty acid yields of E. Coli
cultures harboring tandem ACPs

| | ACP₁ | ACP₂ | Fold- | ACP₃ | Fold |
|---|---|---|---|---|---|
| | (mg/L) | (mg/L) | change | (mg/L) | change |
| 37° C. | 14.6 ± 0.3 | 8.7 ± 1.8 | — | 12.0 ± 0.6 | — |
| 22° C. | 20.9 ± 1.7 | 26.0 ± 0.5 | — | 23.7 ± 3.6 | — |
| 15° C. | 42.3 ± 3.1 | 55.7 ± 8.9 | — | 70.2 ± 9.1 | 1.7 |

What is claimed is:

1. A composition comprising a plurality of linked acyl carrier proteins (ACP), wherein a plurality of ACPs are artificially covalently linked to at least one other ACP by a peptide linker, wherein each linkage has the structure ACP-linker-ACP.

2. The linked ACP of claim 1, wherein
a) the plurality of ACP artificially linked via peptide linkers is a linked tandem ACP comprising a first ACP and a second ACP covalently linked to the linker in a structure ACP-linker-ACP arrangement or;
b) the plurality of ACP artificially linked via peptide linkers to create branched or tree-like like linkages and a second ACP covalently linked to the linker in a structure ACP-linker-ACP arrangement.

3. The linked ACP of claim 1, wherein artificially covalently linked peptide is arranged so that the carboxy terminus of the first APC is covalently linked to the amino terminus of the peptide linker and wherein the carboxy terminus of the linker is covalently linked to the amino terminus of the second APC.

4. The linked ACP of claim 2, wherein the linked tandem ACP are arranged in a multi-domain arrangement.

5. The linked ACP of claim 3, wherein the covalently carboxy terminus of the first APC is covalently linked to the amino terminus of the peptide linker and wherein the carboxy terminus of the linker is covalently linked to the amino terminus of the second APC is repeated.

6. The linked ACP of claim 5, wherein the multi-domain arrangement is a flexible multi-domain arrangement.

7. The ACP of claim 1, wherein the multi domain arrangement is a beads-on-a-string arrangement of domains.

8. The ACP of claim 1, wherein the ACP are produced in a microbial host.

9. The ACP of claim 8, wherein the microbial host is Escherichia coli.

10. The ACP of claim 1, wherein peptide linkers are produced in microorganisms including bacteria, plant, or algae.

11. The ACP of claim 10, wherein the bacteria is a P. profundum.

12. The ACP of claim 1, wherein fatty acid biosynthesis production of tandem linked ACP is increased compared with a single-domain ACP.

13. A method of making artificially linked acyl carrier proteins (ACP) comprising the steps of:
constructing an artificially linked multi domain ACP;
cloning and expressing the artificially linked multi domain ACP; fragments to create artificially linked multi domain ACPs;
purifying a first plurality of artificially linked multi domain ACPs, and
quantifying fatty acid yield of a second plurality of artificially linked multi domain ACPs.

14. The method of claim 13, wherein a plurality of ACP are isolated from a microbial host.

15. The method of claim 13, wherein the microbial host is Escherichia coli.

16. The method of claim 13, wherein a plurality of peptide linkers is isolated from a bacteria.

17. The method of claim 13, wherein the bacteria is P. profundum.

18. The method of claim 13, wherein the artificially linked multi domain ACP fragments are constructed by covalently or non-covalently linking the plurality of ACP genes via the plurality of peptide linker genes.

19. The method of claim 18, wherein artificially covalently liked ACPs has an arrangement wherein the carboxy terminus of the APC covalently linked to the amino terminus of the peptide linker and wherein the carboxy terminus of the linker is covalently linked to the amino terminus of a second APC.

20. The method of claim 19, wherein the artificially covalently linked peptide is arranged so that the carboxy terminus of the first APC is covalently linked to the amino terminus of the peptide linker and wherein the carboxy terminus of the linker is covalently linked to the amino terminus of the second APC.

21. The method of claim 18; wherein the artificially linked multi domain ACP fragments are cloned and expressed in a microbial cell to create artificially linked multi domain ACPs.

22. The method of claim 21, wherein the microbial cell is an Escherichia coli cell.

23. The method of claim 18, wherein a pair distribution function (P(r)) of the artificially linked multi-domain ACP is a beads-on-a-string arrangement of domains


SEQUENCE LISTING (8 sequences)

SEQ ID NO: 1 - DNA, 25 bp, Escherichia coli
caccatgagcactatcgaagaacgc

SEQ ID NO: 2 - DNA, 31 bp, Escherichia coli
tacattttgtgtagacgcctggtggccgttg

SEQ ID NO: 3 - DNA, 35 bp, Escherichia coli
aagttcaaggcacaatgagcactatcgaagaacgc

SEQ ID NO: 4 - DNA, 19 bp, Escherichia coli
ttacgcctggtggccgttg

SEQ ID NO: 5 - DNA, 22 bp, Escherichia coli
tctacacaaaatgtagcgattc

SEQ ID NO: 6 - DNA, 20 bp, Escherichia coli
tgtgccttgaacttgtgctg

SEQ ID NO: 7 - protein, 28 aa, Photobacterium Profundum
STQNVAIQTAAPVASASNGLDAAQVQGT

SEQ ID NO: 8 - protein, 126 aa, Escherichia coli
MAILGLGTDIVEIARIEAVIVRSGERLARRVLSDNEWEIWKTHHQPVRFLAKRFAVKEAAAKAFGTGIRNGLAFNQFEVFNDELGKPRLRLWGEALKLAEKLGVANMHVTLADERHYACATVIIES